(19) United States
(12) Patent Application Publication
(10) Pub. No.: US 2012/0253345 A1
(43) Pub. Date: Oct. 4, 2012

(54) DEVICE AND METHOD TO MEASURE BONE HEALING

(75) Inventor: John J. Wixted, Northborough, MA (US)

(73) Assignee: UNIVERSITY OF MASSACHUSETTS

(21) Appl. No.: 13/499,366

(22) PCT Filed: Oct. 6, 2010
(86) PCT No.: PCT/US10/51608

§ 371 Date: Jun. 6, 2012

Related U.S. Application Data
(60) Provisional application No. 61/249,160, filed on Oct. 6, 2009.

(51) Int. Cl. A61B 17/88

(52) U.S. Cl. 606/60; 606/102

(57) ABSTRACT

Advances in our understanding of the cellular mechanisms underlying skeletal regeneration have not been effectively translated in vivo treatment because non-invasive monitoring of bone fracture healing is limited to imaging technology (i.e., for example, x-rays) that cannot be quantified and are subjectively interpreted. The method disclosed herein assesses rates of hip fracture healing using a strain gauge device implanted into a standard orthopedic implant. It has been demonstrated that such a device can measure differences between intact and partially osteotomized fracture models (p<0.05) and that the device can distinguish between stable and unstable fracture patterns in completely osteotomized models across a physiologic range of loads. Such devices are compatible with in vivo bone fracture healing methods, wherein the device is placed onto an orthopedic implant and the strain data is transmitted on a real time basis, thereby providing a non-invasive quantification of bone fracture repair rates.

### FIELD OF INVENTION

The present invention is related to the field of bone fracture healing. In particular, bone fracture healing may be monitored by a device that provides real time data regarding the time-to-union of a bone fracture. Such a device may be integrated with an orthopedic device such that the device measures strain data across a bone fracture. As the method shows that the measured strain decreases, asymptotically to zero, a clinician may evaluate the rate and completion of bone healing. Alternatively, the device may be used in a screening method to evaluate pharmaceutical therapy in animal bone healing models.

### BACKGROUND

Recent studies indicate that over 6 million fractures are reported annually in the United States alone. Skeletal fragility from osteoporosis is widely recognized as a looming public health issue, and with the aging of our population fractures of the shoulder, wrist, spine and hip which are commonly associated with osteoporosis are conservatively expected to double by 2030. (1).

The implication of the growing incidence of skeletal fragility has not gone unnoticed by the pharmacologic industry and a growing body of basic science and clinical translational research has been accumulating over many years. (3,4) The advent of modern techniques of cell biology have facilitated the search for pharmacological targets of the signaling pathways which govern skeletal repair, regeneration, and turnover. (2) Consequently, as the knowledge base increases regarding fracture repair mechanism, the options for pharmacological intervention increase in proportion.

For example, a growing number of animal studies, typically using a mouse model, examine the pharmacologic effects of numerous agents on fracture repair. (3-5) Notably, the translation from animal to human application is well known to be problematic. Many systemic pharmacologic studies have been conducted in animals, but were subsequently shown to be ineffective in humans. (6).

This problem is especially true in the field of bone healing and/or regeneration. While mechanical testing may be useful in determining healing in animal studies, these techniques are not compatible with human clinical trial ethics. Currently, clinicians primarily limited to measuring human bone healing using x-rays, or other non-invasive imaging techniques. These approaches have significant disadvantages because radiographs cannot be readily quantified, and rely on expert opinion panels for analysis. Further, such processes are inherently biased, and require long-term large patient enrollment in studies in order to account for variations in fracture pattern, x-ray technique, and intra-observer differences, to have any degree of accuracy. This requires multiple centers, many patients, long follow up, and is both expensive and potentially biased.

What is needed in the art is a sensing system, placed within an existing implant, which can be implanted under the skin and read remotely, which quantifies how much strain is in the hardware and can be used to quantify the rate at which fractures heal.

### SUMMARY OF THE INVENTION

The present invention is related to the field of bone fracture healing. In particular, bone fracture healing may be monitored by a device that provides real time data regarding the time-to-union of a bone fracture. Such a device may be integrated with an orthopedic device such that the device measures strain data across a bone fracture. As the method shows that the measured strain decreases, asymptotically to zero, a clinician may evaluate the rate and completion of bone healing. Alternatively, the device may be used in a screening method to evaluate pharmaceutical therapy in animal bone healing models.

In one embodiment, the present invention contemplates a device comprising a load sensing element, wherein said element is configured perpendicular to an axis of an orthopedic implant. In one embodiment, the orthopedic axis comprises a longitudinal axis. In one embodiment, the orthopedic axis comprises a lateral axis. In one embodiment, the load sensing element further comprises a wireless data transmitter.

In one embodiment, the present invention contemplates a method comprising: a) providing; i) an orthopedic implant capable of treating a fracture; ii) a half bridge strain gauge configured to be placed into the implant; b) placing the implant across the fracture; c) placing the gauge into the implant, wherein the implant is perpendicular to the implant axis; and d) measuring strain across the implant with the gauge. In one embodiment, the measuring further comprises a plurality of serial strain measurements. In one embodiment, the method further comprises step (e) observing fracture healing, wherein a decreasing differential is determined between the serial strain measurements. In one embodiment, the fracture healing is complete wherein no differential is determined between the serial strain measurements.

In one embodiment, the present invention contemplates a system comprising: a) a half bridge strain gauge capable of reading strain perpendicular to the axis of the implant; and b) an orthopedic implant capable of treating a fracture, wherein the strain gauge is placed into the implant. In one embodiment, the strain sensor system further comprises a wireless sensing device, wherein the strain data is transmitted from the strain gauge to a receiving module for processing.

In one embodiment, the present invention contemplates a method comprising: a) providing; i) a non-human animal exhibiting a bone fracture; ii) a strain gauge sensor system compatible with the non-human animal; and iii) a test compound; b) implanting the strain gauge sensor system in the non-human animal, wherein the system is capable of measuring strain perpendicular to the fracture; c) administering the test compound to said animal; and d) monitoring the rate of change in strain perpendicular to the fracture. In one embodiment, the test compound comprises a pharmaceutical drug. In one embodiment, the test compound comprises a hormone. In one embodiment, the test compound comprises a peptide. In one embodiment, the test compound comprises a vehicle control. In one embodiment, the method further comprises comparing the rate of change strain between a vehicle control and the test compound.

### Definitions

The term “strain gauge” as used herein refers to any device capable of determining the amount of strain between two objects (i.e., an inherent tendency to separate). For example, some strain gauges take advantage of the physical property of electrical conductance's dependence on the electrical conductivity and geometry of a conductor. When an electrical conductor is stretched within the limits of its elasticity such that it does not break or permanently deform, it will become narrower and longer, changes that increase its electrical resistance end-to-end. Conversely, when a conductor is compressed such that it does not buckle, it will broaden and shorten, changes that decrease its electrical resistance end-to-end. From the measured electrical resistance of the strain gauge, the amount of applied stress may be inferred.

The term “orthopedic implant” as used herein, refers to any surgically placed device capable of improving the recovery of a skeletal injury (i.e., for example, a bone fracture). For example, an orthopedic implant may be an intramedullary nail that is placed within the bone marrow cavity that provides strength and immobility to a skeletal injury.

The term “fracture” as used herein, refers to any medical condition in which there is a break in the continuity of the bone. Such a break may be partial or complete. While many fractures are the result of high force impact or stress, bone fracture can also occur as a result of certain medical conditions that weaken the bones, such as osteoporosis, certain types of cancer, or osteogenesis imperfecta, where the fracture is then termed pathological fracture.

The term “time to facture union” as used herein, refers to the length of time from placement of the sensor system to when the measured strain value begins to stabilize. This stabilization demonstrates that the bone segments have fused, thereby forming a union, after which complete healing may occur.

### BRIEF DESCRIPTION OF THE FIGURES

FIG. 1A presents one embodiment of a strain gauge compatible with the present invention that has been placed into an orthopedic implant (i.e., for example, a helical blade of a Synthes Trochanteric Fixation Nail). The wires may be in communication with a sensing system (i.e., for example, a system commercially available from Microstrain, Inc.).

FIG. 1B presents one embodiment of a testing set up to measure simulated bone fracture strain.

FIG. 1C presents one embodiment of a wireless transmission device to measure in vivo bone fracture strain. Transmitter unit. Inducing coil. Strain gauge device. Receiver.

FIG. 2A presents exemplary data showing the load response measured across various simulated bone fractures of differing severity (i.e., complete, two thirds, one third, control).

FIG. 2B presents exemplary data showing averaged load response data measured across various simulated bone fractures of differing severity (i.e., complete, two thirds, one third, control).

FIG. 2C presents exemplary data showing mean peak load based on fracture configuration (i.e., severe intertroke, less severe intertroke).

FIG. 3 presents exemplary data showing the linear relationship between the strain/load ratio between the various simulated bone fractures of differing severity (i.e., complete, two thirds, one third, control).

### DETAILED DESCRIPTION OF THE INVENTION

The present invention is related to the field of bone fracture healing. In particular, bone fracture healing may be monitored by a device that provides real time data regarding the time-to-union of a bone fracture. Such a device may be integrated with an orthopedic device such that the device measures strain data across a bone fracture. As the method shows that the measured strain decreases, asymptotically to zero, a clinician may evaluate the rate and completion of bone healing. Alternatively, the device may be used in a screening method to evaluate pharmaceutical therapy in animal bone healing models.

### I. Translation of Animal Bone Fracture Models to Human Clinical Practice

Translational research may be based on a premise that advances in basic science can be carried forward and developed into practical improvements in clinical treatment. Generally, this involves at least two steps: i) advances in understanding basic biologic processes must be made, and ii) treatments designed to take advantage of this new knowledge must be proven to be safe and effective in humans. Unfortunately, while the knowledge of cellular biology underlying fracture repair has grown tremendously in recent years, very few treatments have been successfully translated into clinical practice. This is due in large part to the difficulty conducting human trials of fracture healing.

If translational research in fracture repair is to keep pace with advances in basic science, an improved metric for quantifying fracture repair in humans must be developed. In one embodiment, the present invention contemplates a method measuring changes in strain across a fracture site, thereby quantifying a bone fracture healing rate. Although it is not necessary to understand the mechanism of an invention, it is believed that when a bone is broken and treated with an implant to stabilize the fracture, the implant is doing “work”. In other words, the implant is resisting forces which would displace the unhealed fracture. For example, intramedullary (IM) hip implants are generally designed to resist shear while allowing for compression. It is further believed that, strain in the implant is highest immediately after implantation, and most importantly, the rate at which the strain seen by the implant decreases might serve as a measure of the rate of fracture repair.

In one embodiment, the present invention contemplates a device comprising a standard IM nail configured with a strain gauge along the central axis of a proximal fixation device. Although it is not necessary to understand the mechanism of an invention, it is believed that such a configured device would be able to measure strain accurately in a bone fracture (i.e., for example, an extra capsular proximal femoral fracture). The data presented herein demonstrates that prototype testing of such a device distinguished between various amounts of healing as simulated by a series of incomplete osteotomies and measured strain across a physiologically relevant range. In some embodiments, the device measures strain that is independent of the relative position of two parts of the IM device. In some embodiments, the device is capable of allowing for transcutaneous readings.

### II. Non-Invasive Bone Fracture Monitoring

It has been reported that strain gauges have been used in orthopedic implants to measure intra-skeletal dynamic forces, such as spine articulation. The present invention contemplates a device and method to measure time-to-union of a bone fracture.

Currently, there are no reported means to accurately quantify time-to-union for a bone fracture. The current gold standard for assessing union is biplanar radiographs, which are not accurate and difficult to compare. Furthermore, x-rays provide limited data because it is impractical for safety and logistical reasons to take daily x-rays, to determine time to union for a fracture. In one embodiment, the present invention contemplates a device capable of measuring strain across a fracture site. In one embodiment, the measured strain decreases as the facture heals. In one embodiment, the device identifies a healed fracture when the measured strain reaches a steady state.

In some embodiment, the disclosed system can be used to quantify time-to-fracture union more accurately than plain radiography, making it a useful tool for clinical monitoring of fracture healing. Although it is not necessary to understand the mechanism of an invention, it is believed that because the device measures time-to-union more accurately, fewer patients will need to be studied for a shorter time, simplifying clinical studies of fracture repair. Consequently, an advantage of the presently disclosed device and method over those currently available is that the improved accuracy results in faster clinical evaluation and reduced medical costs. Such advantages will facilitate the development of pharmaceutics and well as orthopedic implant design technology.

A. Device Development: Simulated Bone Fracture Application

The data presented herein demonstrates the feasibility of a device comprising a strain gauge coupled with a standard cephalomedullary device designed to allow for quantification of fracture healing over time. The IM device was chosen because of the reproducibility and frequency of proximal femoral fractures, standardization of post-operative weight bearing protocols, ease of implantation, and limited modifications needed to the device. The data presented suggest that the contemplated strain gauge/IM nail device could be used in large animal in vivo testing. Such a device quantifies rates of fracture healing, thereby simplifying efforts at translational research to verify methods of enhancing fracture repair.

1. Osteotomized Versus Intact Specimens

Specimens with a complete osteotomy (Group 1), ⅔ osteotomy (Group 2), ⅓ osteotomy (Group 3), and intact specimens (Group 4) were loaded from 0 to 600 pounds in accordance with Study I in Example I. The strain across the implant was measured, and load versus strain curves were generated. Using a correlation curve, the collected strain readings from the device were then expressed as load. By expressing this as load, the relative amounts of load carried by the implant and the bone model could be determined; this represents the percent load sharing by the nail.

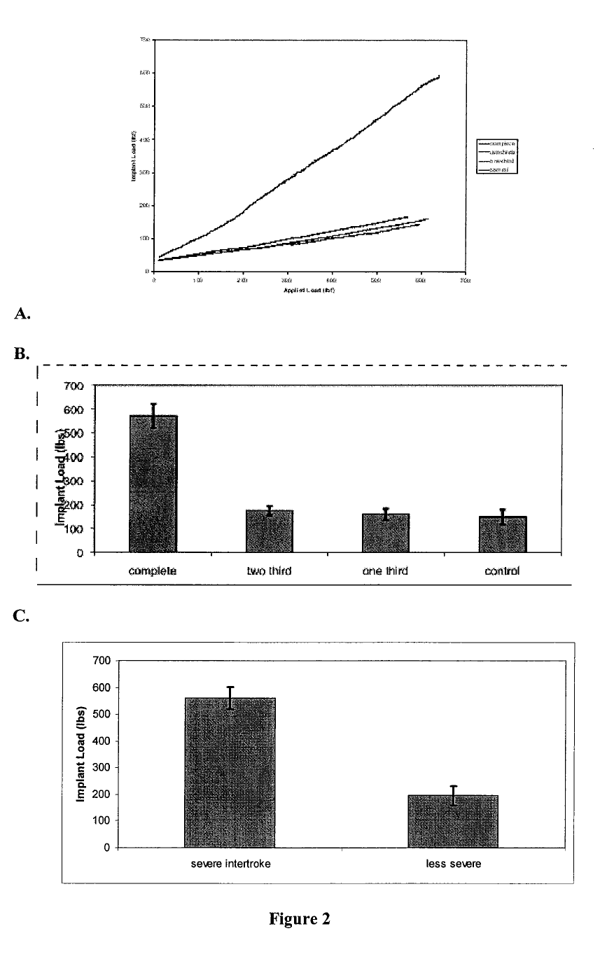

Group 1 specimens (complete osteotomy) registered the highest applied loads, while Group 4 (intact specimens), registered the lowest applied loads. Little difference was seen between Group 2 (⅔ osteotomy), Group 3 (⅓ osteomy), and Group 4 (intact). See, FIG. 2.

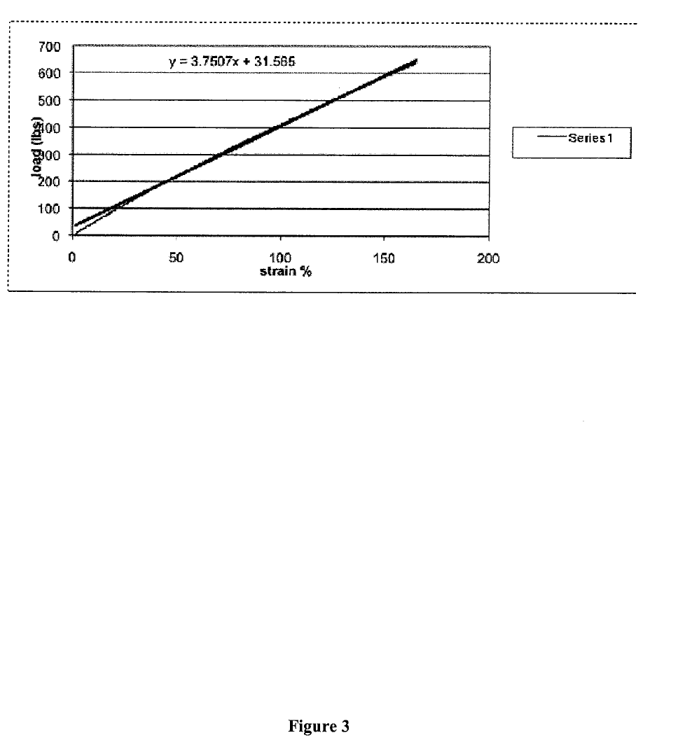

Mean loads were calculated for each group and compared. Statistically significant differences (p<0.05) were seen when comparison was made between the fully ostetomized group and all other groups. While the difference between Group 2 and Group 4 (⅔ vs intact) approached significance (p=0.053), no difference was seen between Group 2 vs Group 3 (⅔ vs ⅓) or Group 3 and Group 4 (⅓ vs intact). See, FIG. 3, and Table 1.

TABLE I
Significance Relationships Between
Simulated Bone Fracture Groups

| Mean Loads, | | | |
|---|---|---|---|
| p value | Group 2 | Group 3 | Group 4 |
| Group 1 vs | p < 0.001 | p < 0.001 | p < 0.001 |
| Group 2 vs | * | p = 0.204 | p = 0.053 |
| Group 3 vs | * | * | p < 0.256 |

Thus the strain gauge/orthopedic device was easily able to discriminate the fully osteotomized group from the partially osteomized groups and the control group. However, the device was less sensitive in discriminating between the partially osteomized groups and the control group.

2. Fracture Pattern Differentiation

In order for the device to be clinically useful, it should be able to characterize the initial stability of a fracture, so that subjects could be grouped accurately for compassion purposes prior to any healing. To simulate this, two groups of complete osteotomies were created. High shear angle osteotomies were cut such that the inclination angle from the horizon was approximately 70 degrees. Low shear angle osteotomies had an inclination angle from the horizontal of approximately 40 degrees. See, Example I, Studies 3 & 4. The device was then inserted and tested from 0 to 600 pounds according to protocol. Mean loads were calculated and compared The device was able to distinguish between the two patterns (i.e., a 70 degree versus 40 degree inclination angle fracture) with a high degree of significance (p<0.001). Such exemplary data shows an averaged load response data measured across different simulated bone fracture patterns. (i.e., 70 degree inclination fracture; 40 degree inclination fracture).

3. Strain Gauge Position Independence

An IM nail was fixed to the mechanical test frame and testing was undertaken without an implant present, wherein a 10 pound pre-load was applied to minimize any motion present between the blade and the nail. A helical blade was then loaded in two conditions: i) the nail was maximally inserted into the blade; and ii) the nail was minimally inserted.

The load/strain curves generated were observed to be indistinguishable. Such exemplary data showing strain/load relationships between two positions of a helical strain gauge blade. (i.e., maximally inserted into the implant, and minimally inserted into the implant). Thus, strain in the blade is likely to be independent of the position of the blade relative to the nail, and changes in strain seen after changes in position of the blade would likely reflect the relative load sharing of the bone, and not intrinsic changes in the load/strain relationship of the sensor.

4. Wireless Transmission Of Measured Strain Data

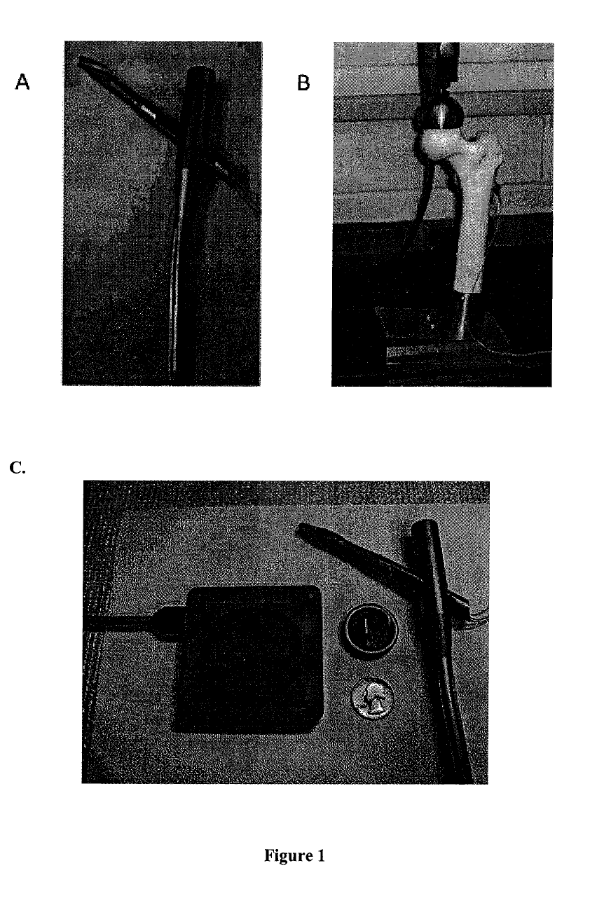

Final testing of the device was performed using a wireless sensing system wherein a transmitter unit and an inducing coil unit were concentrically configured. See, FIG. 1C. The device was able to transmit to the base unit at 5 mm, 10 mm. and 15 mm separation (data not shown). After testing from 10 to 600 pounds, the wireless device was removed and the strain gauge attached directly to the strain channel on the load frame. Output from each device was normalized to maximum output at 600 pounds. Load/strain curves were then generated and placed on the same axis. The curves were nearly identical and showed a high degree of correlation, thus confirming that the wireless sensor was accurate transmitting strain to the base unit at 15 mm of separation between the transmitter and the coil.

In this pilot study, we hypothesized that a standard IM nail could be fitted with a strain gauge and a wireless transmitter to measure strain across a common fracture pattern. A similar in vivo approach has been used previously to quantify implant strain at spinal fusion sites to determine if the spine was fully fused (REF). Our goal was fundamentally different, however. The purpose of the device tested in this study is not for measuring whether any individual has healed or not, but rather to develop a new metric for quantifying fracture healing to simplify translational research into novel methods for fracture repair. By accurately quantifying strain in the implant, we theorize that changes in strain over time can be used as a means of quantifying rates of healing.

We are able to demonstrate on the basis of this pilot data that the half bridge strain gauge we used in this study does measure accurately across the expected range of loads. The strain is independent of the relative position of the blade and the nail, which is critical because the blade is designed to allow for a certain range of compression across the fracture site when used to treat fractures. Furthermore, the sensor can accurately discriminate between fully osteotomized and partially osteotomized or intact specimens.

5. In Vivo Applicability

The data presented in the above study demonstrates the feasibility of using changes in implant strain to measure healing in the fracture in vivo. For example, the study determined strain gauge parameters and overall system design to provide a practical and easy method to configure with conventional orthopedic implants. The data demonstrated an inability of the device to differentiate between the ⅔ osteotomy and the ⅓ osteotomy. Although it is not necessary to understand the mechanism of an invention, it is believed that these results are primarily a function of the intact calcar providing a similar degree of load sharing by the bone. It is believed that an alternative osteotomy, for example, made from inferior to superior, that differences between partial osteotomy would be detectable.

Fracture healing in vivo is believed to be dynamic and temporal. Consequently, the incomplete osteotomy patterns studies herein reflect only some of the possible partial fractures. Nonetheless, the observed differences between the above specimens (i.e., for example, a complete osteotomy versus an intact specimens) were highly significant, and it is notable that the differences between ⅔ osteotomy and intact specimens approached statistical significance.

This data would suggest that a strain sensor system would be able to discriminate, in vivo, between “unstable”, i.e. fully osteotomized, and “stable”, i.e. partially healed specimens, and that as fractures become increasingly stable the strain would reach steady state. In some embodiments, the method comprises identifying a fracture callous identified by strain stability. Further in vivo testing may distinguish between whether strain stability reflects “time to stabile configuration” or “time to healing”.

In one embodiment, the strain sensor discriminates between “unstable” and “stable”, and tracks relative healing rates over time. Alternatively, the strain sensor may also detect a fracture collapse as detected by strain stabilization. Fracture collapse and/or fracture compression at the fracture site is commonly seen in clinical practice. Although it is not necessary to understand the mechanism of an invention, it is believed that inherent fracture stability reflects the relative positioning of the fracture fragments thereby affecting fracture healing rates.

In order to differentiate inherent fracture stability, the strain sensor system was demonstrated to differentiate between different fracture patterns. For example, the data presented herein shows data differentiating between high and low shear angle osteotomies. The device was clearly able to discriminate between the patterns. For example, the percent of load sharing in the low shear angle fractures was quite high, while in the higher angle fracture the vertical nature of the osteotomy exhibited far less load sharing. This suggests that the device could indeed identify a group of patients with a “homogeneously stable” fracture pattern after implantation, and before fracture healing.

Lastly, the device contemplated herein can be used in a wireless fashion. Separation distance was tested between the coil and the transmitter to confirm that the wireless transmitter did not adversely affect sensor readings. A similar device has previously been reported in human use for measuring strain following spinal fusion, and the device was able to transmit transcutaneously. The device is small, does not require batteries, it would likely need to be implanted close to the skin.

B. Clinical Therapeutics: In Vivo Bone Fracture Application

Nearly every hip fracture in the US is treated operatively, as the results of operative management are generally superior to treatment by closed or conservative, i.e. non-operative, management. For example, cephalomedullary nails are one example of an orthopedic implant compatible with the present invention that may be used to hold fractured bones (i.e., for example, hip bones) together while they heal. In one embodiment, the present invention contemplates a device comprising a strain sensor system configure with a cephalomedullary nails (i.e., for example, a cephalomedullary nail commercially available from Synthes, Inc.).

The data presented herein illustrate the effectiveness of the strain sensor system/cephalomedullary nail implant combination. However, this data is not meant to be limiting as the strain sensor system is capable of being configured with any type of orthopedic implant device. Cephalomedullary nails were chosen as an illustrative platform for several reasons:

1. Cephalomedullary factures are extremely common thereby facilitating enrolling patients for clinical trials. Furthermore, data regarding successful treatment of a common fracture results in broader appeal for commercial application and development. For example, current studies of cephalomedullary fracture are generally conducted on tibial shaft fractures because the x-rays can be more easily analyzed, despite the fact that these fractures are relatively uncommon, particularly in the elderly osteoporotic population.

2. The strain across a cephalomedullary fracture can be easily quantified by the available prototype technology. For example, intertrochanteric fractures generally occur nearly perpendicular to the implant position, greatly simplifying strain gauge placement.

3. Commercially available cephalomedullary implants require only minor reconfiguration to be compatible with a strain sensor system. Such minor changes do not have a structurally significant effect on the existing hardware design.

### EXPERIMENTAL

### Example I

### Simulated Bone Fracture Strain Measurements

A standard sized half bridge strain gauge designed to measure strain perpendicular to the long axis of a shackle bolt (Strain Sert, Inc) was custom fitted to the central portion of a 110 mm helical blade used in a common TM fixation device (Trochanteric Fixation Nail, Synthes, Inc). Prior to testing any specimens, a IM nail with the helical blade/sensor apparatus inserted through the nail was independently mounted to the frame. See, FIG. 1. The TM nail/gauge device was tested to 650 pounds to generate a calibration curve for correlating strain and load. This curve was linear from approximately 80 lbs to over 600 lbs.

Cortical foam plastic models of the proximal femur (Pacific Research, Inc) were used to simulate bone fractures. All specimens had placement of the IM device consistent with manufacturer's protocol, which was then removed prior to osteotomy on a band saw and reinserted after osteotomy. Proximal femoral osteotomies were completed with groups of n=6 femurs and were begun on the superior aspect of the proximal femur.

All testing was conducted on an electromechanical test frame under load control mode (Admet, Inc). Specimens were loaded at 10 lbs/sec to maximum 6001bs. Load was held for ten seconds and returned to baseline at the same rate. 600 lbs was chosen to simulate estimated expected load across the joint during single leg stance (REF).

Study I: Four groups were compared and consisted of complete osteotomy, ⅔ osteotomy with intact calcar, ⅓ osteotomy with intact calcar, and uncut specimens for control.

Study II: Two groups were compared, and consisted of high shear angle fractures (approximately 70 degrees) and low shear angle fractures (approximately 40 degrees).

Study III: The device was tested on the load frame without specimens. The nail was mounted to the test frame using a custom jig and the tip of the nail was pre-loaded to ten pounds prior to testing to allow for any motion between the nail and the blade to be minimized prior to testing.

Study IV: The device was tested on the load frame without specimens. The nail was mounted to the test frame using a custom jig and the tip of the nail was pre-loaded to ten pounds prior to testing to allow for any motion between the nail and the blade to be minimized prior to testing.

Strain in each sample was recorded, and using a correlation curve was expressed as load. Mean loads and standard deviations were calculated for each group. Given the small sample size, t-distributions were calculated for comparison among groups, with p<0.05 chosen for significance.

We claim:

1. A device comprising a load sensing element, wherein said element is configured perpendicular to an axis of an orthopedic implant.

2. The device in claim 1, wherein said orthopedic axis comprises a longitudinal axis.

3. The device in claim 1, wherein said orthopedic axis comprises a lateral axis.

4. The device in claim 1, wherein said load sensing element further comprises a wireless data transmitter.

5. A method comprising:
a) providing;
i) an orthopedic implant capable of treating a fracture;
ii) a half bridge strain gauge configured to be placed into said implant;
b) placing said implant across said fracture;
c) placing said gauge into said implant, wherein said implant is perpendicular to said implant axis; and
d) measuring strain across said implant with said gauge.

6. The method of claim 5, wherein said measuring further comprises a plurality of serial strain measurements.

7. The method of claim 6, wherein said method further comprises step (e) observing fracture healing, wherein a decreasing differential is determined between said serial strain measurements.

8. The method of claim 6, wherein said fracture healing is complete wherein no differential is determined between said serial strain measurements.

9. A system comprising:
a) a half bridge strain gauge capable of reading strain perpendicular to the axis of the implant; and
b) an orthopedic implant capable of treating a fracture, wherein the strain gauge is placed into the implant.

10. The system of claim 9, wherein said strain sensor system further comprises a wireless sensing device, wherein said strain data is transmitted from said strain gauge to a receiving module for processing.

11. A method comprising:
a) providing;
i) a non-human animal exhibiting a bone fracture;
ii) a strain gauge sensor system compatible with the non-human animal; and
iii) a test compound;
b) implanting the strain gauge sensor system in the non-human animal, wherein the system is capable of measuring strain perpendicular to the fracture;
c) administering the test compound to said animal; and
d) monitoring the rate of change in strain perpendicular to the fracture.

12. The method of claim 11, wherein said test compound comprises a pharmaceutical drug.

13. The method of claim 11, wherein said test compound comprises a hormone.

14. The method of claim 11, wherein said test compound comprises a peptide.

15. The method of claim 11, wherein said test compound comprises a vehicle control.

16. The method of claim 15, wherein method further comprises comparing the rate of change strain between a vehicle control and the test compound.
